(19) United States
(12) Patent Application Publication
(10) Pub. No.: US 2025/0134365 A1
(43) Pub. Date: May 1, 2025

(54) BATTERY SYSTEM FOR A LARYNGOSCOPE

(71) Applicant: COVIDIEN LP, Mansfield, MA (US)

(72) Inventors: Jiangeng Gu, Shanghai (CN); Li Wang, Shanghai (CN); Mingxia Sun, Shanghai (CN); Xiaojun Qian, Shanghai (CN); Chunlei Hou, Shanghai (CN)

(21) Appl. No.: 18/704,825

(22) PCT Filed: Oct. 13, 2021
(86) PCT No.: PCT/CN2021/123513

§ 371 Date: Apr. 25, 2024

(51) Int. Cl. A61B 1/00; A61B 1/267

(52) U.S. Cl. CPC A61B 1/00029; A61B 1/00006; A61B 1/267

(57) ABSTRACT

A battery assembly for a medical device includes a housing that is configured to removably couple to the medical device. The battery assembly also includes a battery within the housing, a first terminal exposed on an outside of the housing, and a power button that forms part of the housing. The battery assembly further includes a battery control circuit within the housing, wherein the battery control circuit is configured to disable an output path from the battery to the first terminal when the power button is in an off position.

### BACKGROUND

The present disclosure relates generally to medical devices and, more particularly, to a rechargeable battery system for a laryngoscope.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

During treatment of a patient, a tracheal tube or other medical device may be used to control a flow of air, food, fluids, or other substances into the patient. For example, the tracheal tube may be used to control the flow of air or other gases through a trachea of the patient and into the lungs of the patient during a mechanical ventilation procedure. A laryngoscope is commonly used to facilitate insertion of the tracheal tube into the trachea of the patient as part of an intubation procedure. A video laryngoscope is a type of laryngoscope that includes an imager, such as a camera, to obtain an image (e.g., moving image) of the airway of the patient during the intubation procedure.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a battery assembly for a medical device includes a housing that is configured to removably couple to the medical device. The battery assembly also includes a battery within the housing, a first terminal exposed on an outside of the housing, and a power button that forms part of the housing. The battery assembly further includes a battery control circuit within the housing, wherein the battery control circuit is configured to disable an output path from the battery to the first terminal when the power button is in an off position.

In one embodiment, a battery system configured to power a medical device includes a battery assembly. The battery assembly includes a housing that is configured to removably couple to the medical device, a battery within the housing, and a first terminal exposed to an outside of the housing. The battery assembly also includes a first switch within the housing, wherein the first switch is configured to move between an open configuration that disables an output path from the battery to the first terminal and a closed configuration that enables the output path from the battery to the first terminal.

In one embodiment, a method of operating a battery system for a medical device includes disabling, with a battery control circuit, an output path from a battery within a housing to a first terminal exposed on an outside of the housing in response to actuation of a power button that forms part of the housing to an off configuration.

### BRIEF DESCRIPTION OF THE DRAWINGS

Advantages of the disclosed techniques may become apparent upon reading the following detailed description and upon reference to the drawings in which:

FIG. 1 is a perspective view of a laryngoscope system, in accordance with an embodiment of the present disclosure;

FIG. 2 is a block diagram of the laryngoscope system of FIG. 1, in accordance with an embodiment of the present disclosure;

FIG. 3 is a perspective top view of a battery assembly that may be used in the laryngoscope system of FIG. 1, in accordance with an embodiment of the present disclosure;

FIG. 4 is a cross-sectional side view of the battery assembly of FIG. 3, in accordance with an embodiment of the present disclosure;

FIG. 5 is a perspective top view of a battery cavity of a laryngoscope that is configured to receive the battery assembly of FIG. 3, in accordance with an embodiment of the present disclosure;

FIG. 6 is a schematic diagram that illustrates operational features of a battery control circuit that may be implemented to provide power from the battery assembly of FIG. 3, in accordance with an embodiment of the present disclosure;

FIG. 7 is a flow diagram of a method of operating the battery control circuit of FIG. 6, in accordance with an embodiment of the present disclosure;

FIG. 8 is a rear perspective view of a battery assembly that may be used in the laryngoscope system of FIG. 1, wherein the battery assembly includes a switch circuit on a flexible printed circuit board (FPCB), in accordance with an embodiment of the present disclosure;

FIG. 9 is a top view of the switch circuit on the FPCB of FIG. 8, in accordance with an embodiment of the present disclosure;

FIG. 10 is a perspective view of a dome switch over the switch circuit on the FPCB of FIG. 8, in accordance with an embodiment of the present disclosure;

FIG. 11 is side view of the dome switch of FIG. 10 over the switch circuit on the FPCB of FIG. 8, in accordance with an embodiment of the present disclosure; and

FIG. 12 is a schematic diagram that illustrates operational features of a battery control circuit that includes the dome switch of FIG. 10 over the switch circuit on the FPCB of FIG. 8, in accordance with an embodiment of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A medical professional (e.g., clinician, operator, user) may use a laryngoscope to view a patient's oral cavity to facilitate insertion of a tracheal tube (e.g., endotracheal tube, tracheostomy tube, or transtracheal tube) into the patient's trachea during an intubation procedure. Certain laryngoscopes may provide an indirect view of the patient's oral cavity via prisms, lenses, reflective surfaces, or the like. However, it is presently recognized that it may be desirable to position a camera on a portion of the laryngoscope that is inserted into the patient's oral cavity to obtain an image (e.g., moving image or video) of the patient's oral cavity. The image may then be displayed on a display screen during the intubation procedure to enable the medical professional to visualize the patient's oral cavity and to facilitate insertion of the tracheal tube.

Accordingly, the disclosed embodiments generally relate to a laryngoscope system that includes a laryngoscope with a body (e.g., reusable body) having an elongate portion (e.g., a camera stick) that supports a camera, a display portion with a display screen that is configured to display an image obtained by the camera, and a handle portion with a handle that is configured to be gripped by the medical professional during an intubation procedure. The laryngoscope may be operated with a single hand (such as the medical professional's left hand), while another hand (such as the medical professional's right hand) grips the tracheal tube and guides it forward into the patient's oral cavity. The medical professional may view the images that show advancement of the tracheal tube on the display screen in order to guide the tracheal tube into its proper position. A blade for the laryngoscope is provided as a removable cover (e.g., disposable, reusable, or reprocessable cover or sleeve) that fits over the elongate portion of the body of the laryngoscope. Together, the blade and the elongate portion of the body may form an insertable assembly that is configured to be inserted into and to manipulate the patient's oral cavity.

The laryngoscope system also includes a battery system that provides power to the various components of the laryngoscope, such as to the camera and the display screen. The battery system may enable the laryngoscope to be portable and easy to handle during the intubation procedure (e.g., not connected to an electrical outlet or another remote power source by an electrical cable). The battery system may include a battery assembly that houses a battery (e.g., rechargeable battery) and that is removable (e.g., separable) from the body of the laryngoscope. As discussed herein, the battery assembly includes certain features, such as a battery control circuit, that enable the battery assembly to be deposited into a fluid to be cleaned without draining the battery (e.g., without forming a short circuit through the fluid). Thus, the battery assembly may be efficiently cleaned and reused for multiple intubation procedures.

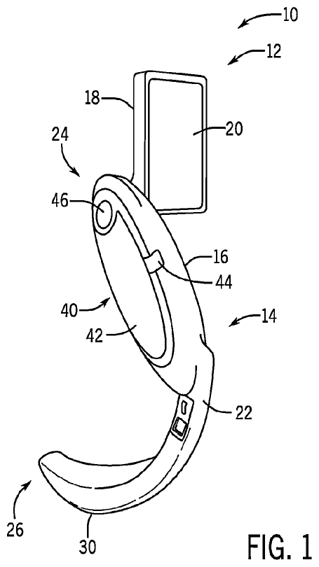

With the foregoing in mind, FIG. 1 is a perspective view of a laryngoscope system 10, in accordance with an embodiment of the present disclosure. The laryngoscope system 10 includes a laryngoscope 12 (e.g., video laryngoscope) with a body 14, which may include a handle portion 16 that is ergonomically shaped as a handle to facilitate grip by a user. The body 14 may also include a display assembly 18 having a display screen 20, and the body 14 may further include a camera stick 22 that supports a camera (e.g., imager) and a light source. As shown, the body 14 extends from a proximal end 24 to a distal end 26. The display assembly 18 may be positioned at or near the proximal end 24 and may extend laterally from the handle portion 16, while the camera stick 22 may be positioned at or near the distal end 26.

It should be appreciated that the display assembly 18 may be formed as an integrated piece with the handle portion 16, such that a housing of the display assembly 18 and an exterior of the handle portion 16 are continuous and/or are formed from the same material. Alternatively, the display assembly 18 may be formed as a separate piece and adhered or otherwise coupled (e.g., fastened) to the handle portion 16. The display assembly 18 may be fixed relative to the handle portion 16 or may be pivotable, such that an angle or a position of the display assembly 18 may be adjusted by the user. The camera stick 22 may also be formed as an integrated piece with the handle portion 16, such that a housing of the camera stick 22 and the exterior of the handle portion 16 are continuous and/or are formed from the same material. Alternatively, the camera stick 22 may be formed as a separate piece and adhered or otherwise coupled (e.g., fastened) to the handle portion 16. In addition to supporting the camera and the light source, the camera stick 22 also supports cables or electrical leads that couple the camera and the light source to electrical components in the handle portion 16 and/or in the display assembly 18 of the body 14.

During use of the laryngoscope 12 to intubate a patient, a removable blade 30 is positioned over the camera stick 22 (e.g., like a sleeve). The blade 30 includes an internal channel or passage that is sized to accommodate the camera stick 22 and to position the camera of the camera stick 22 at a suitable angle to visualize the airway. The passage may terminate at a closed end face, and a field of view of the camera is oriented through the closed end face. The blade 30 is at least partially transparent (such as transparent at the closed end face, or transparent along an entirety of the blade 30) to permit the camera of the camera stick 22 to capture images through the blade 30. An image captured by the camera of the camera stick 22 is displayed on the display screen 20.

The laryngoscope system 10 also includes a battery system 40 that is configured to provide power to various components of the laryngoscope 12, such as to the display screen 20 and the camera of the camera stick 22. The battery system 40 includes a battery assembly 42 that houses a battery (e.g., rechargeable battery) and electrical circuitry (e.g., a battery control circuit). The battery assembly 42 may be configured to couple to the body 14 of the laryngoscope 12, such as to the handle portion 16 of the body 14 of the laryngoscope 12. For example, the battery assembly 42 may be configured to be seated (e.g., via a snap-fit) within a battery cavity formed in the handle portion 16 of the body 14 of the laryngoscope 12. The battery assembly 42 may be removable (e.g., separable) from the body 14 of the laryngoscope 12, such as by exerting a force (e.g., pull force) on a tab 44 of the battery assembly 42.

As discussed in more detail herein, the battery assembly 42 includes certain features that enable the battery assembly 42 to be deposited into a fluid to be cleaned without draining the battery (e.g., without forming a short circuit through the fluid). For example, a power button 46 is configured to control (e.g., enable and disable) an output path of the battery within the battery assembly 42, instead of controlling an ability of the laryngoscope 12 to use the power from the battery. More particularly, the power button 46 is configured to control a switch within the battery assembly 42. In an open position, the switch interrupts the output path of the battery and blocks a voltage signal from reaching a first terminal (e.g., contact; positive) of the battery assembly 42 that is exposed to come into contact with a corresponding contact in the battery cavity of the laryngoscope 12. Thus, when the switch is in the open position, the battery assembly 42 is not able to form a complete circuit to output power when an electrical load (e.g., an electrical component, such as the laryngoscope 12) is in proper contact with the first terminal and a second terminal (e.g., contact; negative) of the battery assembly 42. Similarly, when the switch is in the open position, the battery assembly 42 is not able to form a short circuit to output power when a fluid connects the first terminal and the second terminal of the battery assembly 42. In this way, the battery assembly 42 may be deposited into the fluid to be cleaned and may be reused for multiple intubation procedures over time.

It should be appreciated that the laryngoscope system 10 may include or may be configured to communicate (e.g., via wired or wireless connections) with other devices or systems, such as a monitor (e.g., medical monitor). Thus, the laryngoscope 12 may also include a communication device (e.g., transmitter or transceiver) that is configured to provide information to and/or receive information from the other devices or systems. The laryngoscope 12 may also include processing circuitry (e.g., one or more processors and a memory device) that carries out various processing steps, such as processing the image data from the camera of the camera stick 22 and instructing display of the images on the display screen 20, for example. The battery system 40 may provide power to any components of the laryngoscope 12, including the communication device and/or the processor circuitry, as well as the display screen 20 and the camera of the camera stick 22.

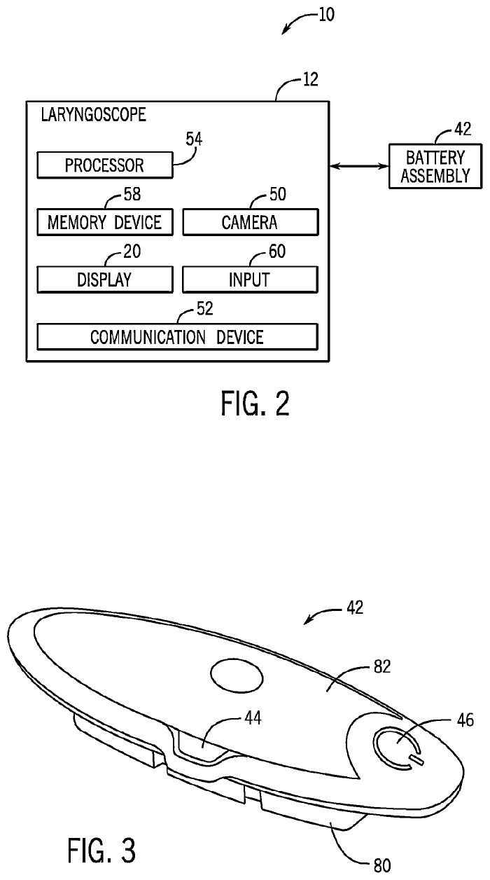

FIG. 2 is a block diagram of the laryngoscope system 10, in accordance with an embodiment of the present disclosure. As shown, the laryngoscope system 10 includes the laryngoscope 12 with the display screen 20 and various other components, such as a camera 50, a communication device 52, one or more processors 54, a memory device 58, and/or one or more user input devices 60. As shown, the battery assembly 42 is configured to be a removable component of the laryngoscope 12.

The one or more processors 54 may process signals, such as signals generated by the camera 50 and/or control signals provided via inputs, such as the one or more input devices 60. The one or more processors 54 may be used to execute software. For example, the one or more processors 54 may receive signals from the camera 50 and execute software to generate an image and/or to carry out any of a variety of processes in accordance with the present disclosure (e.g., display the image). The one or more processors 54 may include multiple microprocessors, one or more “general-purpose” microprocessors, one or more special-purpose microprocessors, and/or one or more application specific integrated circuits (ASICS), or some combination thereof. For example, the one or more processors 54 may include one or more reduced instruction set (RISC) processors. The memory device 58 may include a volatile memory, such as random access memory (RAM), and/or a nonvolatile memory, such as read-only memory (ROM). It should be appreciated that the memory device 58 may include flash memory, a hard drive, or any other suitable optical, magnetic, or solid-state storage medium, other hardware memory, or a combination thereof. The memory device 58 may store a variety of information and may be used for various purposes. For example, the memory device 58 may store processor-executable instructions (e.g., firmware or software) for the one or more processors 54 to execute, such as instructions for processing signals generated by the camera 50 to generate the image, provide the image on the display screen 20, and/or store the image. The memory device 58 may store data (e.g., previously obtained images, time data), instructions (e.g., software or firmware for generating the images, storing the images, transmitting the images), and any other suitable data. The communication device 52 is configured to provide information to and/or to receive information from other devices or systems. It should be appreciated that other processors and/or memory devices disclosed herein (e.g., as part of a battery control circuit) may have any of the features of the one or more processors 54 and the memory device 58, respectively.

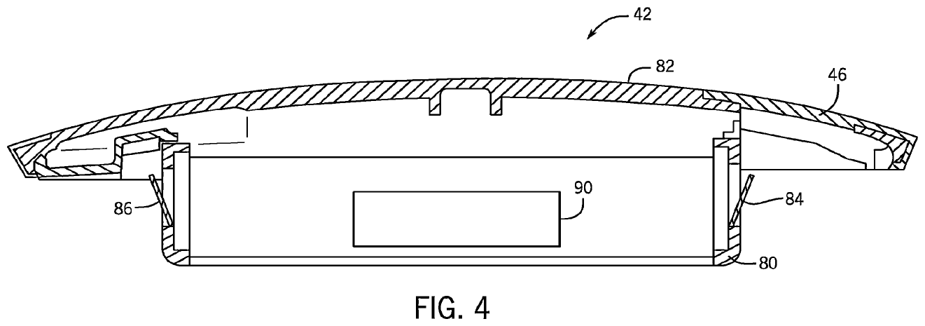

FIG. 3 is a perspective top view of the battery assembly 42 that may be coupled to the body 14 of the laryngoscope 12 shown in FIG. 1, in accordance with an embodiment of the present disclosure. Additionally, FIG. 4 is a cross-sectional side view of the battery assembly 42 of FIG. 3, in accordance with an embodiment of the present disclosure. As shown in FIG. 3 and FIG. 4, the battery assembly 42 includes a battery housing 80, which may couple to the body 14 of the laryngoscope 12. In some embodiments, the battery housing 80 may couple to the handle portion 16 of the body 14 of the laryngoscope 12. In such cases, the battery housing 80 may effectively form part of the handle portion 16 of the body 14 of the laryngoscope 12 shown in FIG. 1. The battery housing 80 may include an outer shell 82 that has a curvature to facilitate grip by the user during the intubation procedure. However, it should be appreciated that the battery assembly 42 and the battery housing 80 may have any suitable shape and/or may couple to any suitable portion of the body 14 of the laryngoscope 12.

Regardless of its shape or form, the battery housing 80 may include the power button 46 that is configured to adjust between an “off” position or input and an “on” position or input via contact/pressure applied to the power button 46 by the user. The power button 46 is configured to control the output path of the battery within the battery housing 80. More particularly, the power button 46 is configured to control the switch within the battery housing 80 to control the output path of the battery within the battery housing 80. When the power button 46 is in the off position, the switch is in the open position to disable the output path such that the battery cannot provide a voltage signal through a first terminal 84 (e.g., contact; battery terminal; positive). That is, when the power button 46 is in the off position, the battery within the battery housing 80 cannot provide power via the first terminal 84 and a second terminal 86 (e.g., contact; battery terminal; negative) even if corresponding contacts within the battery cavity of the laryngoscope 12 are in proper contact with the first terminal 84 and the second terminal 86. Similarly, when the power button 46 is in the off position, the battery within the battery housing 80 cannot provide power via the first terminal 84 and the second terminal 86 even if a fluid connects the first terminal 84 and the second terminal 86 (e.g., cannot be short circuited when submerged in fluid). In this way, the battery housing 80 can be deposited into the fluid to be cleaned and reused for multiple intubation procedures.

However, when the power button 46 is in the on position, the switch is in a closed position to enable the output path of the battery within the battery housing 80 such that the battery is can provide the voltage signal through the first terminal 84. Thus, when the power button 46 is in the on position, the battery within the battery housing 80 can provide power via the first terminal 84 and the second terminal 86 to the laryngoscope 12. Notably, when the power button 46 is in the on position (e.g., inadvertently), the battery within the battery housing 80 may be able to provide power via the first terminal 84 and the second terminal 86 even if a fluid connects the first terminal 84 and the second terminal 86 (e.g., may be able to be short circuited when separated from the laryngoscope 12 and submerged in fluid). However, as discussed in detail herein, the battery assembly 42 may also include electrical circuitry that is configured to detect a presence of the laryngoscope 12 (e.g., receive feedback from the laryngoscope 12) and to disable the output path of the battery within the battery housing 80 in response to failure to detect the presence of the laryngoscope 12.

In this way, the power button 46 acts directly on the switch within the battery housing 80 to interrupt and to connect the output path of the battery within the battery housing 80 and operates as an on-off switch for the output path of the battery within the battery housing 80, instead of acting on circuitry within the laryngoscope 12 to operate as an on-off switch for delivery and usage of the power within the laryngoscope 12. Thus, actuation of the power button 46 to the on position connects components within the battery housing 80 to one another (e.g., connects the first terminal 84 to a voltage source) and activates the battery system 40 to enable the battery within the battery housing 80 to provide power to the laryngoscope 12. Furthermore, actuation of the power button 46 to the off position disconnects components within the battery housing 80 from one another (e.g., disconnects the first terminal 84 from the voltage source) and deactivates the battery system 40 to block the battery within the battery housing 80 from providing power to the laryngoscope 12. As shown in schematic form in FIG. 4 and discussed in more detail herein, a battery control circuit 90 is supported within the battery housing 80 to enable certain features of the disclosed techniques.

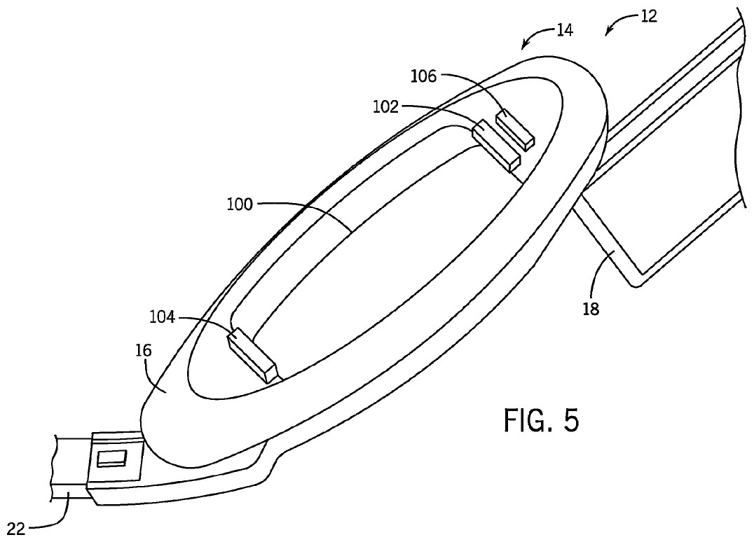

FIG. 5 is a perspective top view of a battery cavity 100 within the handle portion 16 of the body 14 of the laryngoscope 12, in accordance with an embodiment of the present disclosure. The battery cavity 100 is configured to receive and to couple (e.g., removably couple; via a snap-fit) to the battery housing 80. As shown, the battery cavity 100 includes a first contact 102 (e.g., laryngoscope contact) and a second contact 104 (e.g., laryngoscope contact). When the battery housing 80 is properly seated within the battery cavity 100, the first terminal 84 contacts the first contact 102 and the second terminal 86 contacts the second contact 104. However, while the power button 46 is in the off position, the battery within the battery housing 80 cannot provide (e.g., is blocked from providing) power via the first terminal 84 and the second terminal 86 (e.g., the first terminal 84 is isolated from the voltage source). Then, once the power button 46 is actuated to the on position, the output path of the battery within the battery housing 80 is enabled such that the battery can provide power via the first terminal 84 and the second terminal 86.

In some embodiments, actuation of the power button 46 to the on position may be sufficient to provide the power from the battery to the laryngoscope 12, and then the battery may continue to provide the power to the laryngoscope 12 until actuation of the power button 46 to the off position or until the battery is depleted. However, it is presently recognized that it may be desirable for the battery control circuit 90 to also include processing circuitry that is configured to control an additional switch within the battery housing 80 to enable the output path of the battery and to interact with (e.g., receive feedback from) a power on contactor 106 of the laryngoscope 12 to provide more advanced battery control and power supply features.

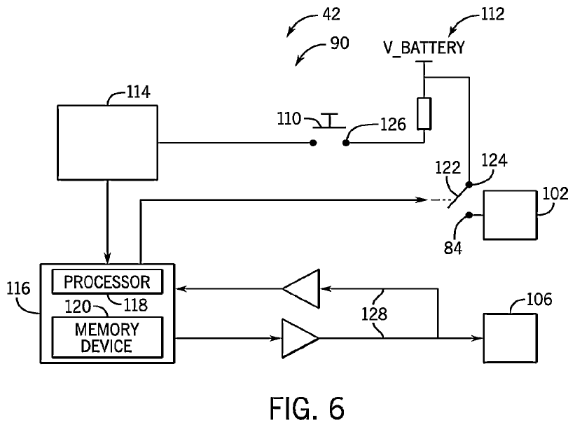

FIG. 6 is a schematic diagram that illustrates operation of the battery control circuit 90 that may be implemented as part of the battery assembly 42, in accordance with an embodiment of the present disclosure. As shown, the battery control circuit 90 includes a first switch 110 (e.g., power button switch), a battery 112 (e.g., a voltage source), a power unit 114, a battery controller 116 (e.g., electronic controller), and a second switch 122 (e.g., additional switch; controller switch). The battery controller 116 may include processing circuitry, such as one or more processors 118 and a memory device 120, that enables the battery controller 116 to execute a power-on protocol, as described herein.

As shown, the first switch 110 is positioned between the battery 112 and the power unit 114, and the power unit 114 is configured to convey power (e.g., voltage common collector [VCC] power) to the battery controller 116. In operation, actuation of the power button 46 (e.g., adjustment to the on position) closes the first switch 110, which causes the power unit 114 to power the battery controller 116. In response to receipt of the power at the battery controller 116 (which indicates to the battery controller 116 that the power button 46 is in the on position), the battery controller 116 actuates the second switch 122 to connect or to enable the output path of the battery 112 (e.g., to connect the battery 112 to the first terminal 84, such as via an intermediate contact 124). Then, as long as the battery assembly 42 is properly seated in the battery cavity 100, the voltage signal from the battery 112 will pass through the connection between the first terminal 84 and the first contact 102 to provide power to the laryngoscope 12. With reference to FIG. 6, the intermediate contact 124 (e.g., drive signal contact) and an intermediate contact 126 (e.g., detection contact) are both connected to the battery 112 and are both disconnected from downstream circuitry (e.g., from the power unit 114 and the battery controller 116, and from the first terminal 84, respectively) while the power button 46 is in the off position. Generally, the intermediate contact 124 and the intermediate contact 126 are both connected to the battery 112 and are both connected to the downstream circuitry while the power button 46 is in the on position, with some exceptions in certain embodiments as discussed herein.

In some embodiments, upon receipt of the power, the laryngoscope 12 (e.g., via the one or more processors 54 and the power on contactor 106) may provide an indication of appropriate power signal properties (e.g., power-on protocol, voltage level, and/or pulse width) for the laryngoscope 12 to the battery controller 116. In particular, the power that is received at the laryngoscope 12 may cause the laryngoscope 12 to transmit feedback (e.g., a feedback signal; an electronic signal) to the battery controller 116 via the power on contactor 106 and a separate signal line 128 (or lines) that electrically connects the power on contactor 106 to the battery controller 116 when the battery housing 80 is properly seated in the battery cavity 100 of the laryngoscope 12. The battery controller 116 may then provide an on/off driving signal to the power on contactor 106 that is appropriate for the laryngoscope 12 and that effectively activates (e.g., turns on) the power on contactor 106 to enable the laryngoscope 12 to receive and to use the power from the battery 112, such as to operate the video features of the laryngoscope 12.

Notably, if the power button 46 is actuated to the on position (e.g., inadvertently) while the battery assembly 42 is not properly fitted into the battery cavity 100 (e.g., does not contact the first contact 102, the second contact 104, and/or the power on contactor 106), the battery controller 116 does not receive the indication from the power on contactor 106 of the laryngoscope 12. In the absence of the indication from the power on contactor 106, the battery controller 116 may open the second switch 122 to disable the output path and to stop the delivery of the power from the battery 112 to the laryngoscope 12. In particular, if the battery controller 116 does not receive the indication (e.g., within a period of time, such as within 0.1, 0.2, 0.5, or 1 seconds, of actuation of the power button 46 to the on position), the battery controller 116 may open the second switch 122 to disable the output path of the battery 112. In this way, if the power button 46 is in the on position while the battery assembly 42 is not properly seated within the battery cavity 100 (e.g., the battery assembly 42 is submerged in the fluid during the cleaning procedure), the battery controller 116 may quickly stop or interrupt the output path of the battery to block the possibility of a short between the first terminal 84 and the second terminal 86. In other words, the battery control circuit 90 does not permit a sustained supply of the power from the battery 112 (e.g., does not maintain the output path of the battery 112) unless the battery controller 116 receives the indication from the laryngoscope 12, and thus, the indication may act as a confirmation from the laryngoscope 12 that the laryngoscope 12 is present and coupled to the battery assembly 42 in an appropriate and expected manner.

In some embodiments, the battery control circuit 90 may be simplified compared to the battery control circuit 90 shown in FIG. 6. For example, the power unit 114, the battery controller 116, and the second switch 122 may be omitted. In such cases, the first switch 110 may connect the battery 112 to the first terminal 84 (e.g., connect an intermediate contact to the first terminal 84) when the first switch 110 is in the closed position to thereby enable the output path of the battery 112. In this way, the actuation of the power button 46 to the on position may be sufficient to provide the power to the laryngoscope 12, and then the battery may continue to provide the power to the laryngoscope 12 until actuation of the power button 46 to the off position or until the battery is depleted. As another example, the laryngoscope 12 may not be configured to provide the feedback via the power on contactor 106 and/or the battery controller 116 may not be configured to receive or to use the feedback from the power on contactor 106. In such cases, the battery controller 116 may facilitate delivery of the power from the battery 112 to the laryngoscope 12 by controlling the second switch 122 (e.g., based on any of a variety of factors or as part of other power-on techniques) and/or by providing an on/off driving signal to the power on contactor 106 (e.g., a standard or set driving signal that is not customized based on the feedback from the laryngoscope 12).

It should also be appreciated that instead of or in addition to an electronic signal that conveys the appropriate power signal properties via the power on contactor 106, the indication or the feedback may be any other electronic signal or mechanical interface that confirms the presence of the laryngoscope 12 and/or a type of the laryngoscope 12. For example, the battery controller 116 may be configured to detect a threshold resistance in the circuit (e.g., that indicates that the laryngoscope 12 is coupled to the battery 112, rather than merely a fluid) and/or to receive an indication (e.g., from a presence sensor) of a presence of the power on contactor 106 or some other feature of the laryngoscope 12. In this way, the battery controller 116 may initiate and/or maintain the output path of the battery 112 of the battery assembly 42 when (e.g., only when) there is an indication that the battery assembly 42 is properly coupled to the laryngoscope 12. In any case, these features (e.g., the feedback or confirmation) may advantageously enable the battery assembly 42 to be used with multiple different types of laryngoscopes 12 (e.g., different models) that have different appropriate power signal properties (e.g., different power requirements or preferred power input settings). Indeed, these features may enable the battery assembly 42 to be used with multiple different types of medical devices, including different types of laryngoscopes 12 and/or medical devices other than laryngoscopes 12 that have different appropriate power signal properties.

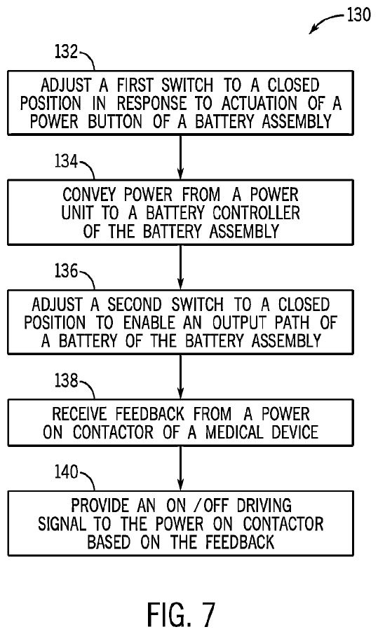

FIG. 7 is a flow diagram of a method 130 of operating the battery control circuit 90 of FIG. 6, in accordance with an embodiment of the present disclosure. The method 130 disclosed herein includes various steps represented by blocks. It should be noted that at least some steps of the method 130 may be performed as an automated procedure by a system, such as the battery system 40. Although the flow chart illustrates the steps in a certain sequence, it should be understood that the steps may be performed in any suitable order and certain steps may be carried out simultaneously, where appropriate. Additionally, steps may be added to or omitted from of the method 130.

In step 132, the first switch 110 moves to the closed position in response to actuation of the power button 46 to the on position. In step 134, the power unit 114 conveys power to the battery controller 116, which indicates a position of the power button 46 to the battery controller 116 (in this way, the battery controller 116 may be considered to detect the position of the power button 46 and respond accordingly). In step 136, the battery controller 116 instructs movement of the second switch 122 to the closed position to connect or to enable the output path of the battery 112 (e.g., to connect the battery 112 to the first terminal 84, such as via the intermediate contact 124). Then, as long as the battery assembly 42 is properly seated in the battery cavity 100, the battery 112 is able to power the laryngoscope 12 via the respective connections between the first terminal 84 and the first contact 102 and the second terminal 86 and the second contact 104.

In step 138, the battery controller 116 receives feedback (e.g., an electronic signal) via the power on contactor 106 and a separate signal line (or lines) that electrically connects the power on contactor 106 to the battery controller 116 when the battery housing 80 is properly seated in the battery cavity 100 of the laryngoscope 12. In step 140, the battery controller 116 may then provide an on/off driving signal to the power on contactor 106 that is appropriate for the laryngoscope 12 and that effectively activates (e.g., turns on) the power on contactor 106 to enable the laryngoscope 12 to receive and to use the power from the battery 112, such as to operate the video features of the laryngoscope 12. As discussed herein, the battery control circuit 90 provides many advantages, such as blocking the output path of the battery 112 via actuation of the power button 46 to the off position to thereby enable the battery assembly 42 to be submerged in fluid to be cleaned without draining the battery 112.

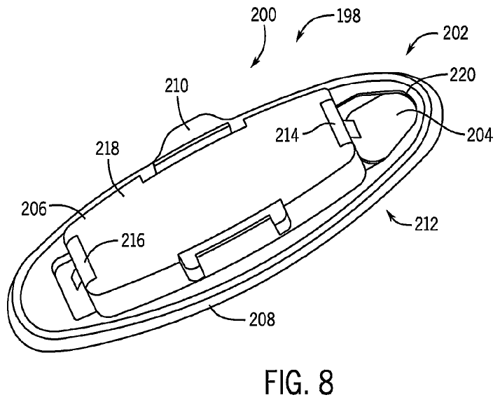

It is also presently recognized that other features may enable a battery assembly for the laryngoscope 12 to be submerged in fluid for cleaning. With the foregoing in mind, FIGS. 8-12 illustrate and describe features of a battery assembly that includes a switch circuit on a flexible printed circuit board (FPCB). In particular, FIG. 8 is a rear perspective view of a battery system 198 that includes a battery assembly 200 that may be used in the laryngoscope 12 of FIG. 1, wherein the battery assembly 200 includes a switch circuit 202 on a FPCB 204, in accordance with an embodiment of the present disclosure.

The battery assembly 200 includes a battery housing 206, which may couple to the body 14 of the laryngoscope 12. In some embodiments, the battery housing 206 may couple to the handle portion 16 of the body 14 of the laryngoscope 12. In such cases, the battery housing 206 may effectively form part of the handle portion 16 of the body 14 of the laryngoscope 12 shown in FIG. 1. The battery housing 206 may include an outer shell 208 that has a curvature to facilitate grip by the user during the intubation procedure and a tab 210 that facilitates separation of the battery assembly 200 from the body 14 of the laryngoscope 12. In some embodiments, the outer shell 208 and/or the tab 210 have an appearance that is similar to or the same as the outer shell 82 and the tab 44 of the battery housing 80 of the battery assembly 42 of FIG. 3. However, it should be appreciated that the battery assembly 200 and the battery housing 206 may have any suitable shape and/or may couple to any suitable portion of the body 14 of the laryngoscope 12. Additionally, the battery assembly 200 also includes a power button 212, which may have an appearance and/or an operation that is similar to or the same as the power button 46 of the battery assembly 42 of FIG. 3.

The battery assembly 200 is configured to provide power via a first terminal 214 (e.g., contact; battery terminal; positive) and a second terminal 216 (e.g., contact; battery terminal; negative). In particular, when the battery assembly 200 is properly seated in the battery cavity 100, respective connections form between the first terminal 214 and the first contact 102 and the second terminal 216 and the second contact 104. As discussed in more detail herein, the switch circuit 202 on the FPCB 204 is part of a water-resistant switch that facilitates submersion of the battery assembly 200 in fluid for cleaning. The switch circuit 202 on the FPCB 204 may be particularly useful in view of manufacturing challenges (e.g., expense and time) with respect to achieving a full seal (e.g., a fluid-tight seal) of the battery housing 206. For example, even if it may be feasible to achieve a full seal of a main chamber 218 of the battery housing 206 (e.g., via welding and/or adhesives), it may be difficult or not feasible to achieve a full seal at a recess 220 that supports the switch circuit 202 on the FPCB 204 and/or a flexible cover (e.g., rubber material) that is positioned over the power button 212.

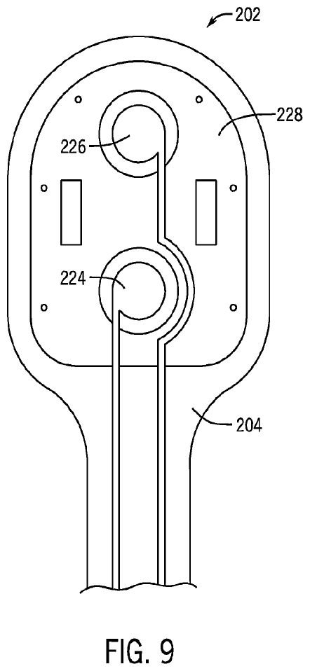

With the foregoing in mind, FIG. 9 is a top view of the switch circuit 202 on the FPCB 204, in accordance with an embodiment of the present disclosure. The switch circuit 202 is printed on the FPCB 204 with three signal networks formed by a first pad 224, a second pad 226, and a third pad 228. The first pad 224 presents a voltage signal from the battery within the battery housing 206, the second pad 226 is connected to the first terminal 214 (e.g., directly or via a switch that is actuated by a battery controller within the battery housing 206), and the third pad 228 presents a negative (or functional ground) of the battery within the battery housing 206.

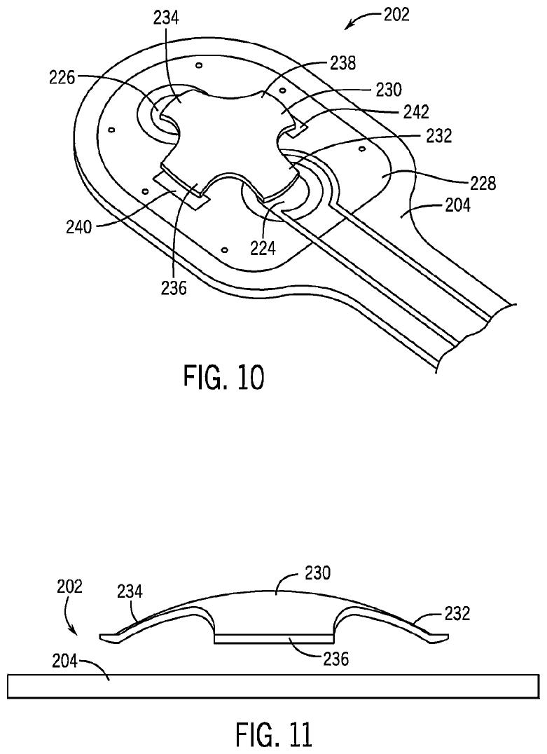

FIG. 10 is a perspective view of a dome switch 230 over the switch circuit 202 on the FPCB 204, and FIG. 11 is side view of the dome switch 230 over the switch circuit 202 on the FPCB 204, in accordance with an embodiment of the present disclosure. The dome switch 230 may be formed of a conductive material (e.g., metal) and positioned under the flexible cover of the power button 212. When the user presses on the flexible cover of the power button 212 to adjust the power button 212 to the on position or input, the dome switch 230 deforms from an open position in which the dome switch 230 does not electrically connect the first pad 224 to the second pad 226 to a closed position in which the dome switch 230 forms a bridge that electrically connects the first pad 224 and the second pad 226, while also bypassing the third pad 228. In the closed position, the dome switch 230 therefore connects the output path of the battery of the battery assembly 200 and enables the battery to provide the voltage signal through the first terminal 214.

As shown, the dome switch 230 may have a first prong 232 that contacts the first pad 224 and a second prong 234 that contacts the second pad 226 when the dome switch 230 is in the closed position. The dome switch 230 may also curve upwardly away (e.g., concave) from the FPCB 204 between the first prong 232 and the second prong 234 to form a dome shape, which enables the dome switch 230 to form the bridge between the first pad 224 and the second pad 226, while also bypassing the third pad 228. The dome switch 230 may also include a third prong 236 and a fourth prong 238 that contact respective portions 240, 242 of a substrate material (e.g., insulating material) of the FPCB 204 when the dome switch 230 is in the closed position.

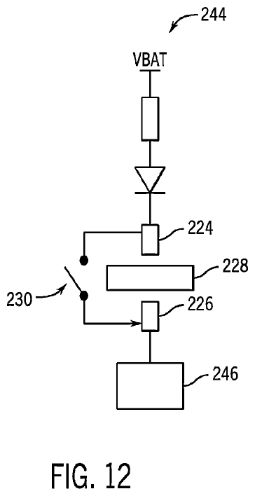

FIG. 12 is a schematic diagram that illustrates operation of the switch circuit 202 on the FPCB 204, in accordance with an embodiment of the present disclosure. As shown, a battery 244 may provide a voltage signal (e.g., across a resistor and amplifier) to the first pad 224. The third pad 228 is positioned to separate (e.g., isolate) the first pad 224 and the second pad 226 from one another. However, upon actuation of the power button 212, the dome switch 230 moves toward the FPCB 204 to the closed position in which the dome switch 230 electrically connects the first pad 224 and the second pad 226, while also bypassing the third pad 228. In this way the dome switch 230 provides the voltage signal from the battery 244 to the second pad 226, which enables the output path of the battery 244 and enables the battery assembly 200 to provide power to the laryngoscope 12. In some embodiments, the second pad 226 may be electrically connected to additional components, such as a battery controller 246 that detects the connection between the first pad 224 and the second pad 226, instructs enabling the output path of the battery 244 (e.g., via additional switches) in response to detecting the connection between the first pad 224 and the second pad 226, interacts with the power on contactor 106 of the laryngoscope 12, such as to receive feedback from the power on contactor 106 of the laryngoscope 12 and/or to provide an on/off driving signal to the power on contactor 106 of the laryngoscope 12 to enable the laryngoscope 12 to receive and to use the power from the battery 244.

Advantageously, the switch circuit 202 on the FPCB 204 blocks the output path of the battery 244 while the power button 212 is in the off position, even if fluid is present at the FPCB 204. For example, even if the battery assembly 200 is submerged in a fluid for cleaning, the fluid cannot form a bridge that crosses over the third pad 228 to transmit the voltage signal from the first pad 224 to the second pad 226. Indeed, not only is the fluid unable to connect the output path of the battery 244, but the fluid may also assist in blocking the output path of the battery 244 because the fluid would likely contact the first pad 224, the second pad 226, and the third pad 228 (e.g., if the power button 212 is inadvertently actuated to the on position while the battery assembly 200 is submerged in the fluid).

The present embodiments provide various techniques and features that facilitate submersion of a battery assembly into a fluid for cleaning, which in turn facilitates reuse of the battery assembly for multiple medical procedures. While certain embodiments relate to use of the battery assembly with a laryngoscope or with various types of laryngoscopes, it should be appreciated that the battery assembly may be used with any of a variety of types of medical devices.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that certain elements of the disclosed embodiments may be combined or exchanged with one another.

What is claimed is:

1. A battery assembly for a medical device, the battery assembly comprising:
a housing that is configured to removably couple to the medical device;
a battery within the housing;
a first terminal exposed on an outside of the housing;
a power button that forms part of the housing; and
a battery control circuit within the housing, wherein the battery control circuit is configured to disable an output path from the battery to the first terminal when the power button is in an off position.

2. The battery assembly of claim 1, wherein the battery control circuit comprises a power button switch that is actuated by the power button and is in an open configuration when the power button is in the off position.

3. The battery assembly of claim 1, wherein the battery control circuit is configured to enable the output path from the battery to the first terminal when the power button is in an on position.

4. The battery assembly of claim 3, wherein the battery control circuit comprises:
a battery controller; and
a power button switch that is actuated by the power button and is in a closed configuration when the power button is in the on position;
wherein, in the closed configuration, the power button switch connects the battery to the battery controller.

5. The battery assembly of claim 4, wherein, in response to connection of the battery to the battery controller, the battery controller instructs a controller switch to connect the battery to the first terminal to enable the output path.

6. The battery assembly of claim 1, wherein the battery control circuit comprises a switch that is in an open configuration when the power button is in the off position, and, in the open configuration, the switch is configured to disable the output path from the battery to the first terminal.

7. The battery assembly of claim 1, wherein the battery control circuit comprises a power button switch and a controller switch, the power button switch is configured to be manually actuated via user contact with the power button, and the controller switch is configured to be electrically actuated via a battery controller.

8. The battery assembly of claim 7, wherein the battery control circuit is configured to disable the output path from the battery to the first terminal when either the power button switch or the controller switch is in an open configuration, and the battery control circuit is configured to enable the output path from the battery to the first terminal when both the power button switch and the controller switch are in a closed configuration.

9. The battery assembly of claim 1, wherein the battery control circuit is configured to receive a feedback signal from the medical device and to provide a drive signal to a power on contactor of the medical device.

10. The battery assembly of claim 9, wherein the battery control circuit is configured to provide the drive signal based on the feedback signal.

11. The battery assembly of claim 10, wherein the feedback signal is indicative of appropriate power signal properties for the medical device, and the drive signal is provided with the appropriate power signal properties for the medical device.

12. The battery assembly of claim 1, wherein the medical device comprises a video laryngoscope.

13. The battery assembly of claim 12, wherein the housing is configured to form part of a handle portion of the video laryngoscope while the battery assembly is coupled to the video laryngoscope.

14. The battery assembly of claim 1, wherein battery control circuit comprises a switch circuit on a flexible printed circuit board and a dome switch that is actuated by the power button.

15. A battery system configured to power a medical device, the battery system comprising:
a battery assembly comprising:
a housing that is configured to removably couple to the medical device;
a battery within the housing;
a first terminal exposed on an outside of the housing; and
a first switch within the housing, wherein the first switch is configured to move between an open configuration that disables an output path from the battery to the first terminal and a closed configuration that enables the output path from the battery to the first terminal.

16. The battery system of claim 15, wherein the battery assembly comprises a power button that forms part of the housing, wherein the first switch is actuated by the power button, is in the open configuration when the power button is in the off position, and is in the closed configuration when the power button is in the on position.

17. The battery system of claim 15, wherein the battery assembly comprises a battery controller within the housing, wherein, in the closed configuration, the first switch enables the output path from the battery to the first terminal by providing power to the battery controller.

18. The battery system of claim 15, wherein the battery control circuit is configured to receive a feedback signal from the medical device and to provide a drive signal to a power on contactor of the medical device.

19. The battery system of claim 15, comprising the medical device, wherein the medical device is a video laryngoscope and comprises a battery cavity that is configured to receive the housing of the battery assembly.

20. A method of operating a battery system for a medical device, the method comprising:
disabling, with a battery control circuit, an output path from a battery within a housing to a first terminal exposed on an outside of the housing in response to actuation of a power button that forms part of the housing to an off configuration.
